(19) United States
(12) Patent Application Publication
(10) Pub. No.: US 2010/0119569 A1
(43) Pub. Date: May 13, 2010

(54) SYSTEMS FOR IMPROVING MATERIAL EXCHANGE WITH AN IMPLANT

(75) Inventor: Nicanor I. Moldovan, Dublin, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 12/521,594

(22) PCT Filed: Dec. 29, 2006
(86) PCT No.: PCT/US06/49612

§ 371 Date: Dec. 17, 2009

(51) Int. Cl. A61K 9/00; A61K 35/12; A61P 43/00

(52) U.S. Cl. 424/422; 424/93.7

(57) ABSTRACT

A system for implanting a device within a biological target region for exchange of material with the biological target region is provided. The system includes an implantable device and a plurality of precursor cells. The implantable device has at least one recess on a first surface of the device, and a filter configured to allow communication between the at least one recess and an internal portion of the device. The at least one recess is configured to receive the precursor cells and to allow the cells to mature and convert into microvessels disposed along the recesses.

### BACKGROUND OF THE INVENTION

Implants are known to be useful for a variety of purposes such as, for example, controlled-release drug delivery, tissue or bone engineering, and cardiovascular applications. When in use, such implants, which may be manufactured from a variety of materials, may cause undesirable side affects or create other problems following implantation into the body of a living organism. Implantation is by its nature an invasive procedure and access to the tissue is created during implantation. The produced wound and its consequent healing may limit integration of the implant in the body. Recipient immune system rejection, excessive scarring, and restenosis are problems frequently encountered with the use of such devices.

In applications in which a device is implanted for delivering materials, such as with a drug delivery device, or receiving materials, such as with a biosensor, an organism's physiological reactions to the introduction of a foreign object may produce conditions that interfere with the transmission of material, such as biological agents, to or from the implanted device, a phenomenon often referred to as “bio-fouling.” Examples of such conditions include the formation of a non-specific protein coat on the implanted device, macrophage interrogation, frustrated phagocytosis with giant cell formation, and the encapsulation of the implanted device by a collagenous fibrous capsule. Thus, there remains a need in the art for improved systems, devices and methods relating to implants.

### SUMMARY OF THE INVENTION

The present application relates to an implantable device. In one embodiment, an exemplary implantable device includes at least one recess of microvascular diameter on a first external surface of the device, and a filter configured to allow communication between the at least one recess and an internal portion of the device. The at least one recess is configured to receive precursor cells and to allow the cells to mature and convert into microvessels disposed along the at least one recess. The internal portion of the device is adapted to receive material for exchange with a biological target region.

The present application also relates to a system for implanting a device within a biological target region for exchange of material within the biological target region. In one embodiment, an exemplary system includes an implantable device and a plurality of precursor cells. The implantable device includes at least one recess of microvascular diameter on a first external surface of the device, and a filter configured to allow communication between the at least one recess and an internal portion of the device. The at least one recess is configured to receive the precursor cells and to allow the cells to mature and convert into microvessels disposed along the at least one recess. The internal portion of the device is adapted to receive material for exchange with a biological target region.

The present application further relates to a method for improving the exchange of material between an implant device and a biological target region. In one exemplary method, an implantable device is provided with at least one recess on a first external surface of the device, and a filter configured to allow communication between the at least one recess and an internal portion of the device. A plurality of precursor cells are deposited in the at least one recess. The precursor cells are allowed to form microvessels along the at least one recess. The device is implanted in the biological target region. The microvessels are allowed to vascularize the target region. Material is exchanged between the internal portion of the device and the biological target region, such that the material passes through the filter and through the microvessels disposed in the at least one recess.

Additional features and aspects of the present invention will become apparent to those of ordinary skill in the art upon reading and understanding the following detailed description of the exemplary embodiments. As will be appreciated, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description of the preferred embodiments given below, serve to explain the principles of the invention.

FIG. 1 schematically illustrates the functioning of an implanted filter-based biomedical device with seeded cell layer;

FIG. 2 schematically illustrates the functioning of an implanted filter-based biomedical device with a substrate surface including recesses for accommodating pre-seeded microvessels;

FIG. 3 schematically illustrates the functioning of an implanted filter-based biomedical device having a film deposited on a substrate surface for accommodating pre-seeded microvessels; and

FIGS. 4A-E schematically illustrate a process of implanting an implantable device including at least one recess for attachment and proliferation of precursor cells for vascularizing a biological target region in which the device is implanted;

### DETAILED DESCRIPTION OF EXEMPLARY EMBODIMENTS OF THE INVENTION

This application relates to a system for improving the exchange of materials between an implanted biomedical device and a biological target region. The exchange of material may involve, for example, delivery of material from an internal portion of the device to the biological target region, such as with a drug or active ingredient delivery device, and/or collection or receipt of material from the biological target region within the device, such as with a biosensor. While the exchange of materials may be the primary function of the implanted device, in other embodiments, this exchange may facilitate other, possibly unrelated functions of the device. For example, an implanted device may deliver angiogenic factors to the biological target region for vascularization of the region.

Various embodiments of the present invention are compatible with a variety of implants types and materials and may be used for multiple therapeutic applications, including, but not limited to: cardiovascular applications (e.g., pacemakers, stents, and vascular prostheses); bone and tissue engineering (e.g., orthopedic: strengthening the interface between a metal implant and bone); mechanical, electrical, or passive subcutaneous implants; implantable drug delivery devices, including controlled-delivery devices; and biosensors. Embodiments of this invention may also be used in most, if not all, situations where seeding, frosting, or coating the exterior of an implant will (i) increase the implant's compatibility with the recipient's biology or physiology; (ii) increase or enhance the performance and/or function of the implant device or implant system; (iii) optimize the tissue healing and response after implantation; or (iv) minimize long term rejection. For most embodiments, the systems and devices described in this application may be assembled using commercially available materials, thereby reducing costs and adding simplicity to the overall process.

According to one aspect of the present application, an implantable device may be provided for exchanging material with a biological target region in which the device is implanted. In one embodiment, the device includes an internal portion configured to receive materials for exchange with the biological target region. For purposes of describing the present invention, the term “exchange with the biological target region” shall be understood as referring to materials that have been received from the biological target region and/or materials to be delivered to the biological target region. The device also includes a filter oriented and configured to allow communication, or passage of material, between the internal portion and an external surface or surfaces of the device. This communication may allow for the exchange of a desired material between the device and the biological target region, while preventing the exchange of other, larger materials, such as contaminants. While the filter may include any porous, permeable, semi-porous, or semi-permeable component, portion, or layer capable of allowing passage of the material to be exchanged, in one exemplary embodiment, the filter includes nanoporous aluminum oxide, with pores of up to 200 nm in diameter, to form a nanofilter for the exchange of particles close to or smaller than the selected pore size. While the filter may include a separate component assembled with the device, in another embodiment, the filter may comprise all or part of a housing or enclosure of the device, which may, for example, be made of a porous material.

A biological target region may respond to the introduction of an implant device with the formation of three layers: a) a thin layer of macrophages and/or giant cells adjacent to the implanted biomaterial ; b) an a vascular or fibrous capsule of about 100 μm containing fibroblasts embedded in a dense collagen mix, and c) an outermost, loosely packed neovascularized region. The fibrous encapsulation, and the macrophages and giant cell formation, can impair the functioning of the implanted device, a phenomenon commonly referred to as “bio-fouling.” For example, vascularization of the device with the surrounding tissue may be impeded, and the exchange of materials between the implanted device and the target region may be obstructed.

To reduce the bio-fouling effects of fibrous encapsulation of the implant device, the external surfaces of the device may be seeded with cells, such as, for example, stem cells, progenitor cells, or mature cells, prior to implantation. This seeding may, for example, be accomplished by magnetic labelling and attachment of cells. An example of such a method is described in co-pending U.S. patent application Ser. No. 11/085,445, filed on Mar. 21, 2005 and published under the PCT as International Pub. No. WO 2005/089507, the entire disclosure of which is incorporated herein by reference. Cell seeding may also be accomplished by coating the implant in a gel-based cell suspension or by other suitable methods. This may produce several advantages. Presumably, uniformly covering the implant device with one or more of these cells types will decrease the formation of fibrous or scar tissue or other blocking formations near or around the implant and may also increase vascularization of the tissue surrounding the device, for improved exchange of materials between the implant device and the target region. Providing precursor cells that have a phenotype similar to that of the host or recipient tissue will presumably limit the amplitude of foreign body immune reaction and will speed recovery following implantation. If the implant device is functioning as a controlled-release device, stimulating and/or differentiating growth or other factors may be included in the formulation being released to enhance the proliferation and/or differentiation of the precursor cells following implantation. Rejuvenation of local tissue cells may also be possible through the use of certain types of progenitor cells attached to the implant.

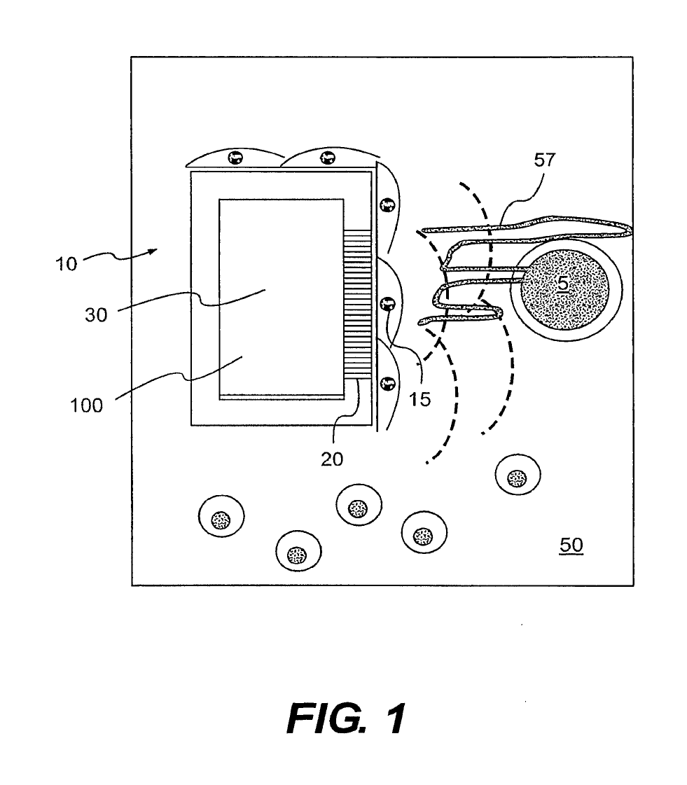

FIG. 1 illustrates a schematic cross-sectional view of a filter-based biomedical delivery device 10 implanted in a biological target region 50, where the device 10 has been coated, seeded or otherwise deposited with biocompatible cells 15 prior to implantation. The cells 15 reduce or eliminate the production of a fibrous capsule around the device, which facilitates delivery of material 100 from the internal portion 30 of the device, through the filter 20, and into the target region. Further, the use of cell growth, differentiation, or other factors, such as, for example, endothelial growth factor (VEGF), either disseminated through the filter 20 from within the device 10 or applied to the surface of the device 10 prior to implantation, may assist in causing the attached cells to proliferate and vascularize the target region 50, forming microvessels 57 extending from the device 10, for more effective delivery of the material 100. However, with this implantation system, non-vascularized attached cells interposed between the device 10 and surrounding microvessels 55, may delay delivery of at least some of the material 100. Also, cells attached prior to implantation of the device may be vulnerable to damage or detachment during or after implantation, due to shear forces resulting from direct contact or friction with the surfaces of the implant, inadequate attachment of the cells to the device, or other conditions to which the cells and/or device may be exposed.

According to aspects of the present application, a surface of an implant device may be configured or adapted to better retain cells to be attached to the device and/or encourage cell differentiation for the formation of microvessels along a patterned first external surface of the device. For example, the surface may be provided with one or more recesses adapted to receive cells brought into contact with the device. This may reduce the likelihood of cell separation from the surface due to shear forces resulting from fluid flow past the device, or other such forces to which the cells may be exposed. It will be understood that any suitable configuration of recesses may be used. In one such example, the surface may be provided with a topography that simulates the desired size and orientation of microvasculature to promote the production and intimate attachment of microvessels along the surface of the device. For example, a surface of an implantable device may be provided with one or more recesses of microvascular size or diameter. The recesses may be configured to receive precursor cells prior to implantation. By limiting cell movement and proliferation in directions lateral to the recesses, the attached cells are stimulated into microvessel formation through cell differentiation.

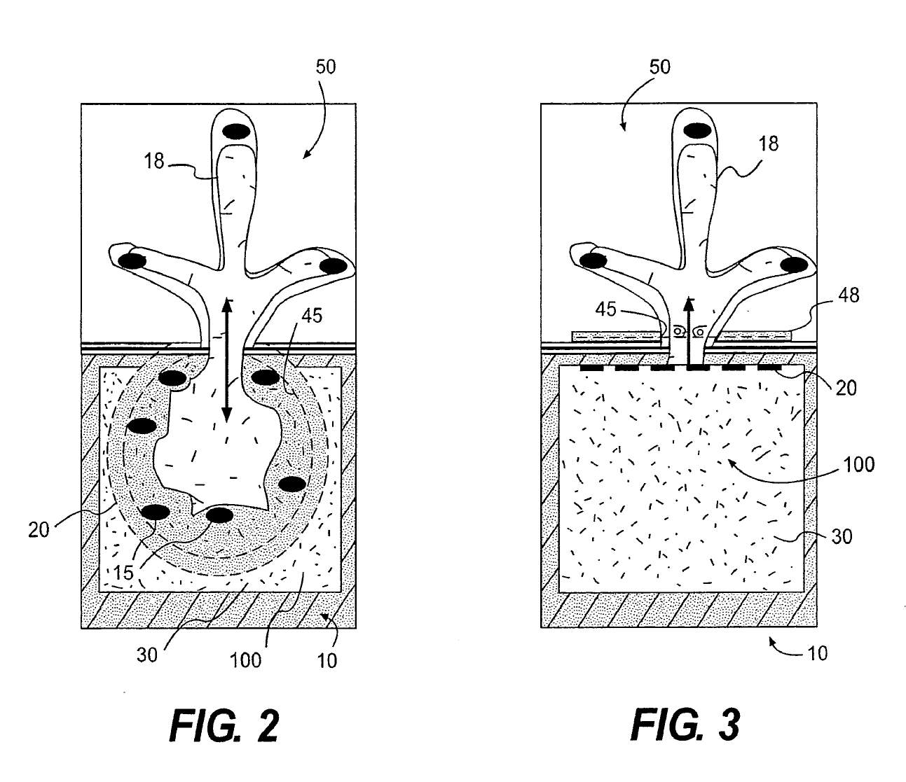

FIG. 2 illustrates a schematic cross-sectional partial view of a micropatterned portion of a filter-based biomedical delivery device 10 implanted in a biological target region 50, where precursor cells 15 have been seeded into a recess 45 prior to implantation and stimulated to form a microvessel extending along the length of the recess, perpendicular to the cross-section. The cells 15 reduce or eliminate the production of a fibrous capsule around the device 10, which facilitates passage or movement of material 100 through the filter 20. Further, the use of cell growth and/or differentiation factors, such as, for example, endothelial growth factor (VEGF), either disseminated through the filter 20 from within the device 10 or applied to the surface of the device 10 prior to implantation, may assist in causing the attached cells 15 to proliferate and differentiate into microvessels 18 to vascularize the target region 50, for more effective delivery of the material 100.

Any suitable method may be used to form a micropattern of one or more recesses on a substrate surface of an implant device. In one embodiment, the recesses are chemically etched onto the surface of the device. In other embodiments, the recesses may be formed by plasma etching, laser writing, or any other suitable form of ablation, or the deposit of material to the surface. The recesses may be formed with a microvascular diameter, which may, for example, range from 5-80 μm, and with a similar corresponding depth, which may, for example range from 20-80 μm. The recesses may substantially cover the entire device, or they may be provided on only a portion of one or more surfaces of the device. While the recess 45 of FIG. 3 is schematically illustrated as circular in cross-section, the recesses may take any cross-sectional shape, such as, for example, a rectangular cross-sectional shape, which may be more easily formed in the substrate. The recesses may also include non-uniform cross sectional shapes. In another embodiment (not shown), portions of the recess or recesses may be fully encased in the substrate material of the implant device to retain and protect the seeded cells and formed microvessels therein.

Any suitable mechanism may be used to attach cells to the micropattern recesses on the surface of the device. As one example, cells to be attached may be suspended in a gel-based matrix, with the gel being applied to the surface or surfaces of the device, by, for example, dipping or rolling the device in the gel, spraying or pouring gel on the device, or other suitable means. As another example, magnetically labeled cells may be magnetically attracted into the micropattern recesses, at least until microvessel formation causes the cells to adhere to the surface of the device without the assistance of magnetic attraction. In such applications, the implantation system may be adapted such that formed microvessels are sufficiently developed to be retained in the recesses before the magnetic attraction between the magnet and the magnetically labeled cells has dissipated or ceased, due to, for example, dilution of the magnetic label caused by cell division, death, and/or detachment; or removal of the magnet from the proximity of the magnetically labeled cells.

In selecting cells for attachment to the micropatterned surfaces of the implant device, in one exemplary application, mature endothelial cells may be chosen both for effective “seeding” of the implant and because after implantation, endothelial cells proliferate and provide enhanced implant vascularization. As described above, enhanced vascularization provides a vessel network that may increase the bioavailability of the implant's drug content. Multiple cell types may be used simultaneously to cover the implant, including mixtures (or layers) of various progenitors, including, for example, tissue-specific cells (bone, cardiac, etc) with non-specific vascular progenitors, seeded together or sequentially on the implant. Genetically engineered cells may also be used and may provide stimulation of neovascularization in peri-implant regions; limitation of the immune/foreign body reaction, correction of the organ functions, or other functions.

According to another inventive aspect of the present application, and as illustrated schematically in FIG. 2, a filter-based implant device 10 may be configured such that the exchange of material through the filter 20 is restricted or limited to the recesses 45 of the external surfaces or substrate of the device 10. By blocking passage of material through the filter to or from a non-patterned second external surface of the device, the exchange of material between an internal portion 30 of the implant device and a target region may be substantially limited to delivery through the cells 15 and microvessels 18 disposed in the recesses 45 of the device 10. When applying this feature to a delivery device, such as a drug or active ingredient delivery device, the material 100 supplied by the implant may be delivered to the target region almost exclusively through the microvasculature, for more direct and effective delivery of the material. When applying this feature to a receiving device, such as a biosensor, the implanted device 10 may be adapted to receive materials almost exclusively through the microvasculature, thereby reducing the introduction of other non-vascular contaminants.

Many different configurations may be used to isolate non-micropatterned surfaces of the implant device from the filter. In one embodiment, a filter may be precisely sized and positioned such that it extends from an internal portion of the device to the micropatterned surfaces of the device only. In another embodiment, the outermost surfaces of the device may be coated or otherwise deposited with a non-permeable material, such as, for example, a polymer or other such coating, such that passage of material through the filter to or from the target region is limited to the recessed portions, which may remain uncoated. In one such embodiment, a non-permeable coating may be applied to a non-micropatterned porous or permeable external surface of an implant device, and one or more recesses may be formed in the non-permeable coating, for example, by photolithography, thereby limiting external exposure of the permeable surface to the patterned portions. A schematic example of such an embodiment is illustrated in FIG. 3, in which the non-permeable layer 48 covers the filter surface 20, except for at the formed recess 45, in which the precursor cells 15 have been seeded and the microvessels 18 have formed.

Any suitable method and mechanism may be used to grow and sustain the attached precursor cells and developing microvasculature on an implant device. As indicated above, in one embodiment, a growth factor may be used to stimulate proliferation of the cells in the recesses. The growth factor may include, for example, vascular endothelial growth factor (VEGF), and may be introduced to the device in a gel-based matrix, such as, for example, Matrigel. In one such embodiment, the precursor cells may be seeded at one portion, such as an end, of a recess or recesses. By filling the grooves with a growth factor, the seeded cells may be stimulated to proliferate along the lengths of the recesses and differentiate to form microvessels within the recesses. It should be noted that the lateral confinement of the micropatterned recesses may inhibit or retard cell proliferation, making the application of a growth factor to the recesses useful in promoting cell growth on the implant.

In another embodiment, the recesses, containing attached precursor cells, may be covered with a protective layer, for example, to protect the attached cells in the recesses from drying or from detachment during implantation. As one example, the device may be coated with a protectant, which may or may not be bioresorbable. The protectant may, for example, possess angiogenic factors to aid in stimulating the formation of microvessels along the micropatterned grooves. In another example, the protectant includes biodegradable polyglycolic acid polymer (PGA). By using a biodegradable or bioresorbable protectant, such as PGA, the protectant may be adapted to degrade and separate from the device after protection of the attached cells is no longer required, which may facilitate vascularization of the biological target region by minimizing interference by protectant covering the implant device.

In another embodiment, a protective cell layer may be seeded around the attached precursor cells disposed in the micropatterned recesses. By seeding the device with a protective cell layer that is biocompatible with the target region, the fibrous encapsulation of the device may be reduced or eliminated, and the attached precursor cells may more easily vascularize the surrounding target region, either incorporating the protective layer cells into the vasculature, or expanding beyond the protective cell layer, which would be more conducive to such expansion than would a fibrous capsule.

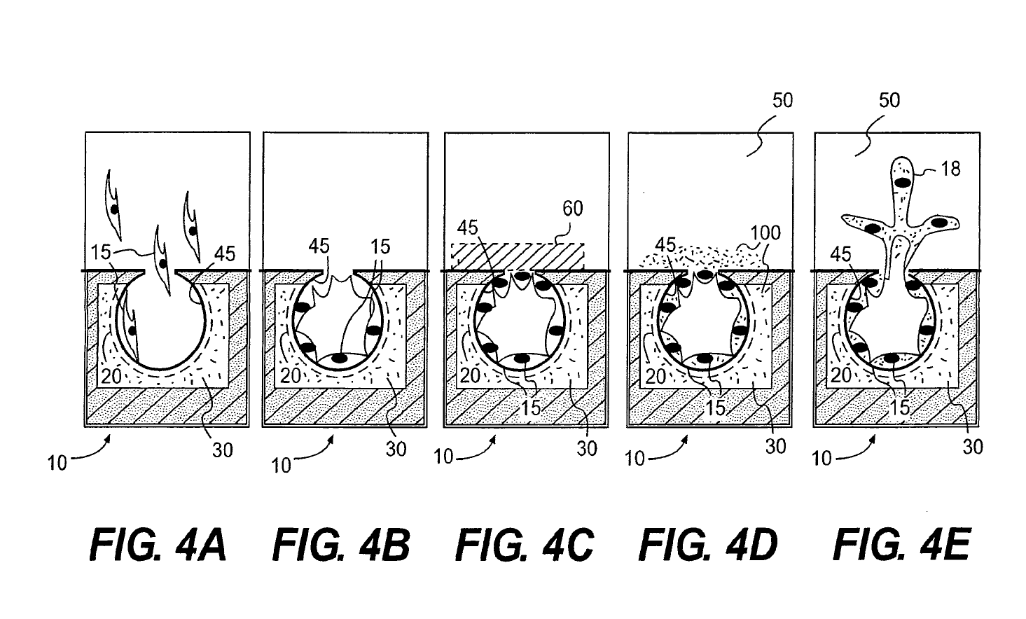

FIGS. 4A-E schematically illustrate an exemplary process of implanting one embodiment of a micropatterned filter-based biomedical device in a biological target region. As shown in FIG. 4A, a plurality of precursor cells 15, such as, for example, endothelial cells, are seeded in the recess or recesses 45 in vitro by a suitable method, such as, for example, application of a gel-based cell suspension, or magnetic attachment of cells to the grooves. As shown in FIG. 4B, the cells 15 are allowed to proliferate and/or differentiate within the recess 45. This proliferation/differentiation may be stimulated or facilitated by the application of a suitable cell growth and/or differentiation factor. As shown in FIG. 4C, the recess 45 with attached cells 15 may be capped or covered by a protective layer 60, such as, for example, a biodegradable protectant or a seeded layer of biocompatible cells. The implant device 10 with protected attached cells or microvessels may then be implanted into the biological target region 50. FIG. 4D illustrates accommodation of the implant device 10 within the target region 50, which may include the release of angiogenic factors 100 through the filter to accelerate microvessel formation. FIG. 4E illustrates expansion or further vascularization of the formed microvessels 18 into the target region 50 for exchange of material 100 between the device 10 and the target region 50. The microvessels 18 may extend from the ends of the recesses 45 and or from the open side along the lengths of the recesses 45.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, devices and components, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

What is claimed:

1. An implantable device comprising:
at least one recess on a first external surface of the device; and
a filter configured to allow communication between the at least one recess and an internal portion of the device,
wherein the at least one recess is configured to receive precursor cells and to allow the cells to mature and convert into microvessels disposed along the at least one recess, and
wherein the internal portion is adapted to receive materials for exchange with a biological target region.

2. The implantable device of claim 1, wherein the filter is further configured to prevent communication between the internal portion of the device and a second external surface of the device.

3. The implantable device of claim 1, wherein the at least one recess is formed in a film layer disposed on at least a portion of the first external surface of the device.

4. The implantable device of claim 1, wherein the first external surface comprises silicon.

5. A system for improving performance of an implantable device, the system comprising:
an implantable device comprising at least one recess on a first external surface of the device, and a filter configured to allow communication between the at least one recess and an internal portion of the device; and
a plurality of precursor cells,
wherein the at least one recess is configured to receive the precursor cells and to allow the cells to mature and convert into microvessels disposed along the at least one recess, and
wherein the internal portion is adapted to receive materials for exchange with the biological target region.

6. The system of claim 5, wherein the implantable device comprises a delivery device, and the internal portion is adapted to deliver the received materials to the biological target region through the filter.

7. The system of claim 6, wherein the delivery device comprises a drug delivery device.

8. The system of claim 5, wherein the implantable device comprises a receiving device, and the internal portion is adapted to receive materials from the biological target region through the filter.

9. The system of claim 8, wherein the receiving device comprises a biosensor.

10. The system of claim 5, further comprising a protectant adapted to cover and protect attached precursor cells in the at least one recess.

11. The system of claim 10, wherein the protectant comprises at least one of a cell growth factor and a cell differentiation factor.

12. The system of claim 10, wherein the protectant comprises angiogenic factors.

13. The system of claim 10, wherein the protectant is biodegradable.

14. The system of claim 5, wherein the precursor cells comprise at least one of stem cells, progenitor cells, mature cells, and genetically engineered cells.

15. A method for improving the exchange of material between an implant device and a biological target region, the method comprising:
providing an implantable device comprising at least one recess on a first external surface of the device, and a filter configured to allow communication between the at least one recess and an internal portion of the device;
depositing a plurality of precursor cells in the at least one recess;
allowing the precursor cells to form microvessels along the at least one recess;
implanting the device in the biological target region;
allowing the microvessels to vascularize the target region; and
exchanging material between the internal portion of the device and the biological target region, wherein the material passes through the filter and through the microvessels disposed in the at lest one recess.

16. The method of claim 15, wherein the filter is further configured to prevent communication between the internal portion of the device and a second external surface of the device.

17. The method of claim 15, wherein exchanging material between the internal portion of the device and the biological target region comprises delivering material from the internal portion to the target region.

18. The method of claim 17, wherein the material delivered from the internal portion to the target region comprises angiogenic factors.

19. The method of claim 15, wherein exchanging material between the internal portion of the device and the biological target region comprises receiving material from the target region into the internal portion.

20. The method of claim 15, further comprising covering the precursor cells disposed within the at least one recess with a protectant.

21. The method of claim 20, further comprising providing at least one of a cell growth factor and a cell differentiation factor in the protectant.

22. The method of claim 20, further comprising allowing the protectant covering the precursor cells to biodegrade, to facilitate vascularization of the target region by the precursor cells.

23. The method of claim 15, wherein providing a device comprising at least one recess of microvascular diameter on a first external surface of the device comprises chemically etching the at least one recess on the first external surface.

24. The method of claim 15, wherein providing a device comprising at least one recess of microvascular diameter on a first external surface of the device comprises chemically etching the at least one recess on the first external surface.

25. The method of claim 16, further comprising seeding a protective cell layer to cover the microvessels formed in the at least one recess, wherein the protective cell layer is biocompatible with the target region.
